(19) United States
(12) Patent Application Publication
(10) Pub. No.: US 2023/0095787 A1
(43) Pub. Date: Mar. 30, 2023

(54) ARTICULATING ULTRASONIC SURGICAL INSTRUMENTS AND SYSTEMS

(71) Applicant: Covidien LP, Mansfield, MA (US)

(72) Inventors: James R. Fagan, Erie, CO (US); Thomas E. Drochner, Longmont, CO (US); Michael B. Lyons, Boulder, CO (US); David J. Van Tol, Boulder, CO (US); Matthew S. Cowley, Frederick, CO (US)

(21) Appl. No.: 17/798,273

(22) PCT Filed: Jan. 28, 2021
(86) PCT No.: PCT/US2021/015398

§ 371 Date: Aug. 8, 2022

Related U.S. Application Data
(60) Provisional application No. 62/983,390, filed on Feb. 28, 2020.

(51) Int. Cl. A61B 17/32

(52) U.S. Cl. CPC A61B 17/320068; A61B 2017/320088; A61B 2017/320074; A61B 2017/320094; A61B 2017/320071; A61B 2017/320098

(57) ABSTRACT

An ultrasonic surgical instrument includes a transducer configured to produce a first mode of ultrasonic energy and a waveguide coupled to the transducer. The waveguide includes a proximal body portion coupled to and extending distally from the transducer. The proximal body portion is configured to receive the first mode of ultrasonic energy from the transducer for transmission therealong. The waveguide further includes a distal body portion, a blade extending distally from the distal body portion, and an articulation portion interconnecting the proximal and distal body portions. The articulation portion is configured to enable articulation of the distal body portion relative to the proximal body portion, to receive the first mode of ultrasonic energy from the proximal body portion, convert the first mode of ultrasonic energy into a second mode of ultrasonic energy, and transmit the second mode of ultrasonic energy along the distal body portion to the blade.

### CROSS-REFERENCE TO RELATED APPLICATIONS

This application is a 371 National Stage Application of International Application No. PCT/US2021/015398, filed Jan. 28, 2021, which claims benefit of U.S. Provisional Patent Application No. 62/983,390, filed Feb. 28, 2020, the entire contents of each of which is hereby incorporated herein by reference.

### FIELD

The present disclosure relates to surgical instruments and systems and, more particularly, to articulating ultrasonic surgical instruments and systems.

### BACKGROUND

Ultrasonic surgical instruments and systems utilize ultrasonic energy, i.e., ultrasonic vibrations, to treat tissue. More specifically, a typical ultrasonic surgical instrument or system includes a transducer configured to produce mechanical vibration energy at ultrasonic frequencies that is transmitted along a waveguide to an ultrasonic end effector configured to treat, e.g., coagulate, cauterize, fuse, seal, cut, desiccate, fulgurate, or otherwise treat tissue.

Some ultrasonic surgical instruments and systems incorporate rotation features, thus enabling rotation of the ultrasonic end effector to a desired orientation within the surgical site. However, even in such instruments and systems, the ability to navigate within the surgical site via rotation and manipulation alone is limited.

### SUMMARY

As used herein, the term “distal” refers to the portion that is being described which is further from a user, while the term “proximal” refers to the portion that is being described which is closer to a user. Further, to the extent consistent, any or all of the aspects detailed herein may be used in conjunction with any or all of the other aspects detailed herein.

Provided in accordance with the present disclosure is an ultrasonic surgical instrument including an ultrasonic transducer configured to produce a first mode of ultrasonic vibration energy and an ultrasonic waveguide coupled to the ultrasonic transducer and extending therefrom. The ultrasonic waveguide includes a proximal body portion coupled to and extending distally from the ultrasonic transducer and configured to receive the first mode of ultrasonic vibration energy from the ultrasonic transducer for transmission therealong. The ultrasonic waveguide further includes a distal body portion, a blade extending distally from the distal body portion, and an articulation portion interconnecting the proximal and distal body portions. The articulation portion is configured to enable articulation of the distal body portion relative to the proximal body portion. The articulation portion is further configured to receive the first mode of ultrasonic vibration energy from the proximal body portion, convert the first mode of ultrasonic vibration energy into a second mode of ultrasonic vibration energy, and transmit the second mode of ultrasonic vibration energy along the distal body portion to the blade.

In an aspect of the present disclosure, the ultrasonic surgical instrument further includes a shaft assembly surrounding at least a portion of the ultrasonic waveguide. The shaft assembly includes an elongated proximal shaft, an articulating section, and a distal support shaft. A jaw is pivotably coupled to the distal support shaft. The proximal body portion of the waveguide extends through the elongated proximal shaft of the shaft assembly, the articulation portion of the waveguide extends through the articulating section of the shaft assembly, the distal body portion of the waveguide extends through the distal support shaft of the shaft assembly, and the blade of the waveguide extends distally from the distal support shaft of the shaft assembly and is positioned to oppose the jaw to enable pivoting of the jaw to clamp tissue between the jaw and the blade.

In another aspect of the present disclosure, the shaft assembly extends distally from a handle assembly. Alternatively, the shaft assembly extends distally from a robotic arm.

In another aspect of the present disclosure, the first and/or second mode is a longitudinal mode, a torsional mode, a transverse mode, or a combination mode.

In yet another aspect of the present disclosure, the articulation portion of the waveguide includes at least one flexure hinge.

In still another aspect of the present disclosure, the articulation portion includes a ball-and-socket joint.

In still yet another aspect of the present disclosure, the articulation portion includes at least one pinned joint.

Another ultrasonic surgical instrument provided in accordance with the present disclosure includes an ultrasonic transducer configured to produce a first mode of ultrasonic vibration energy and an ultrasonic waveguide coupled to the ultrasonic transducer and extending therefrom. The ultrasonic waveguide includes a proximal body portion coupled to and extending distally from the ultrasonic transducer and configured to receive the first mode of ultrasonic vibration energy from the ultrasonic transducer for transmission therealong. The ultrasonic waveguide further includes a distal body portion, a blade extending distally from the distal body portion, and an articulation portion interconnecting the proximal and distal body portions. The articulation portion is configured to enable articulation of the distal body portion relative to the proximal body portion between an aligned position, wherein the proximal and distal body portions are aligned with one another, and at least one angled position, wherein the proximal and distal body portions are disposed at an angle relative to one another.

In the aligned position, the articulation portion is configured to receive the first mode of ultrasonic vibration energy from the proximal body portion and transmit the first mode of ultrasonic vibration energy along the articulation portion and the distal body portion to the blade.

In the at least one angled position, the articulation portion is configured to receive the first mode of ultrasonic vibration energy from the proximal body portion, at least partially convert the first mode of ultrasonic vibration energy into a different mode of ultrasonic vibration energy, and transmit the different mode of ultrasonic vibration energy along the distal body portion to the blade.

In an aspect of the present disclosure, the ultrasonic surgical instrument further includes a shaft assembly surrounding at least a portion of the ultrasonic waveguide. The shaft assembly includes an elongated proximal shaft, an articulating section, and a distal support shaft. A jaw is pivotably coupled to the distal support shaft. The proximal body portion of the waveguide extends through the elongated proximal shaft of the shaft assembly, the articulation portion of the waveguide extends through the articulating section of the shaft assembly, the distal body portion of the waveguide extends through the distal support shaft of the shaft assembly, and the blade of the waveguide extends distally from the distal support shaft of the shaft assembly and is positioned to oppose the jaw to enable pivoting of the jaw to clamp tissue between the jaw and the blade.

In another aspect of the present disclosure, the shaft assembly extends distally from a handle assembly. Alternatively, the shaft assembly extends distally from a robotic arm.

In yet another aspect of the present disclosure, the first mode is one of a longitudinal mode, a torsional mode, a transverse mode, or a combination mode, and the different mode is another one of a longitudinal mode, a torsional mode, a transverse mode, or a combination mode.

In still another aspect of the present disclosure, in at least one of the at least one angled positions, the articulation portion is configured to fully convert the first mode of ultrasonic vibration energy into a second mode of ultrasonic vibration energy as the different mode of ultrasonic vibration energy.

In still yet another aspect of the present disclosure, in at least one of the angled positions, the articulation portion is configured to partially convert the first mode of ultrasonic vibration energy into a blended mode of ultrasonic vibration energy including both the first mode of ultrasonic vibration energy and a second mode of ultrasonic energy as the different mode of ultrasonic vibration energy.

In another aspect of the present disclosure, in a first of the at least one angled positions, the articulation portion is configured to receive the first mode of ultrasonic vibration energy from the proximal body portion, partially convert the first mode of ultrasonic vibration energy into a blended mode of the first mode and a second mode as the different mode of ultrasonic vibration energy, and transmit the different mode of ultrasonic vibration energy along the distal body portion to the blade. In a second of the at least one angled positions, the articulation portion is configured to receive the first mode of ultrasonic vibration energy from the proximal body portion, fully convert the first mode of ultrasonic vibration energy into a second mode as the different mode of ultrasonic vibration energy, and transmit the different mode of ultrasonic vibration energy along the distal body portion to the blade.

In an aspect of the present disclosure, an amount of the at least partial conversion depends on the angle in the angled position. In aspects, the amount of the at least partial conversion is related to the angle in the angled position.

In another aspect of the present disclosure, the articulation portion of the waveguide includes at least one flexure hinge, ball-and-socket joint, and/or pinned joint.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1A is a perspective view of a hand-held articulating ultrasonic surgical instrument provided in accordance with the present disclosure, wherein the elongated assembly is disposed in an un-articulated position;

FIG. 1B is a perspective view of the hand-held articulating ultrasonic surgical instrument of FIG. 1A, wherein the elongated assembly is disposed in an articulated position;

FIG. 2 is a schematic illustration of a robotic surgical system configured for use with an articulating ultrasonic surgical instrument, provided in accordance with the present disclosure;

FIGS. 3-5 illustrate various different articulation portion configurations of an ultrasonic waveguide configured for use with the instrument of FIG. 1A, the system of FIG. 2, or any other suitable surgical instrument or system;

FIG. 6 is a functional block diagram illustrating ultrasonic vibration mode conversion in accordance with aspects the present disclosure; and

FIG. 7 is another functional block diagram illustrating variable ultrasonic vibration mode conversion in accordance with aspects the present disclosure.

### DETAILED DESCRIPTION

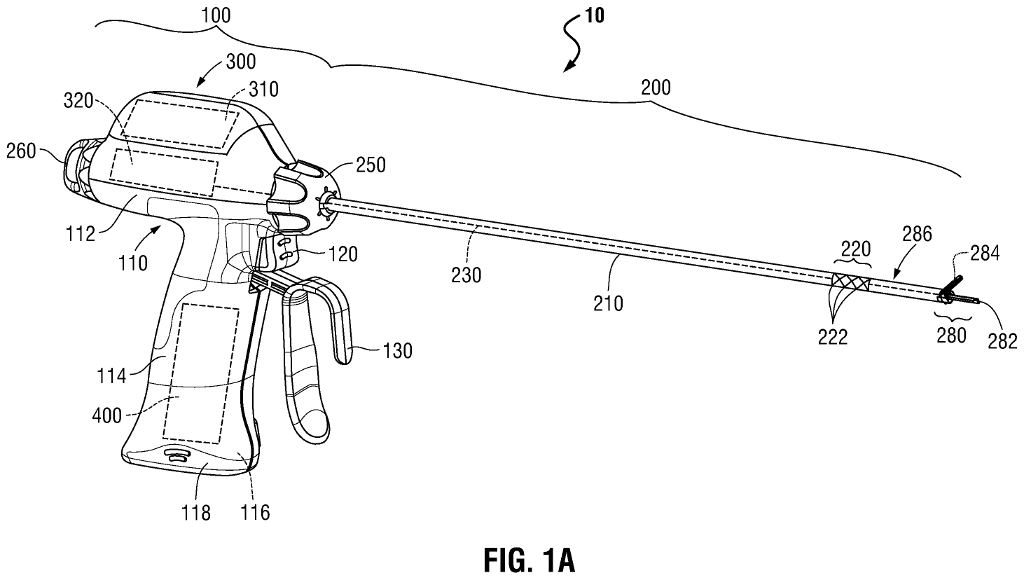

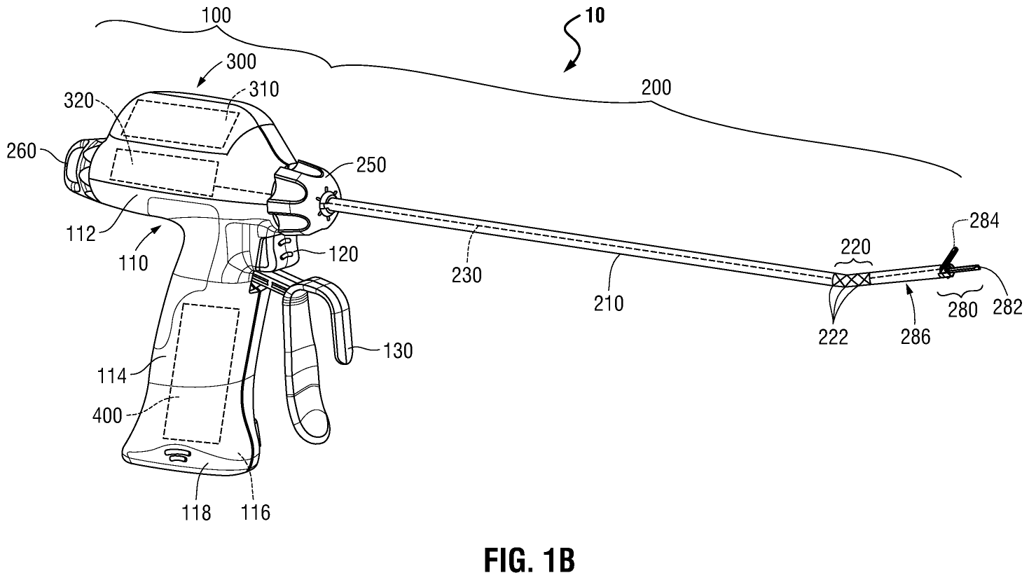

Referring generally to FIGS. 1A and 1B, an embodiment of a hand-held ultrasonic surgical instrument exemplifying the aspects and features of the present disclosure is shown generally identified by reference numeral 10. For the purposes herein, hand-held ultrasonic surgical instrument 10 is generally described. Aspects and features of hand-held ultrasonic surgical instrument 10 not germane to the understanding of the present disclosure are omitted to avoid obscuring the aspects and features of the present disclosure in unnecessary detail.

Hand-held ultrasonic surgical instrument 10 generally includes a handle assembly 100 and an elongated assembly 200 extending distally from handle assembly 100. Handle assembly 100 includes a housing 110 defining a body portion 112 and a fixed handle portion 114. Handle assembly 100 further includes an activation button 120 and a clamp trigger 130.

Body portion 112 of housing 110 is configured to support an ultrasonic transducer and generator assembly (“TAG”) 300 including a generator assembly 310 and an ultrasonic transducer assembly 320. TAG 300 may be permanently engaged with body portion 112 of housing 110 or removable therefrom. Alternatively, generator assembly 310 may be remotely disposed and coupled to ultrasonic surgical instrument 10 by way of a surgical cable.

Fixed handle portion 114 of housing 110 defines a compartment 116 configured to receive a battery assembly 400 and a door 118 configured to enclose compartment 116. An electrical connection assembly (not shown) is disposed within housing 110 of handle assembly 100 and serves to electrically couple activation button 120, generator assembly 310 of TAG 300, and battery assembly 400 with one another when TAG 300 is supported on or in body portion 112 of housing 110 and battery assembly 400 is disposed within compartment 116 of fixed handle portion 114 of housing 110, thus enabling activation of ultrasonic surgical instrument 10 in response to depression of activation button 120. In embodiments where generator assembly 310 is remote from ultrasonic surgical instrument 10, battery assembly 400 and the configuration of fixed handle portion 114 for receiving battery assembly 400 need not be provided, as the remote generator assembly 310 may be powered by a standard wall outlet or other remote power source.

Elongated assembly 200 of ultrasonic surgical instrument 10 includes an elongated shaft 210, an articulation section 220, a waveguide 230 extending through elongated shaft 210 and articulation section 220, a drive assembly (not shown), an articulation assembly (not show), a rotation knob 250, an articulation knob 260, and an end effector 280 including a blade 282, a jaw 284, and a support shaft 286.

Elongated shaft 210 extends distally from body portion 112 of housing 110. Articulation section 220 is coupled to and extends distally from elongated shaft 210 and support shaft 286 of end effector 280 is coupled to and extends distally from articulation section 220. In this manner, articulation of articulation section 220 relative to elongated shaft 210 and housing 110 articulates end effector 280 relative to elongated shaft 210 and housing 110. Articulation section 220 may include one or more articulation components 222, e.g., articulation joint(s), articulation linkage(s), flexible portion(s), etc., coupled between elongated shaft 210 and support shaft 286 of end effector 280 to enable articulation of end effector 280 relative to elongated shaft 210 and housing 110 in at least one plane, e.g., pitch articulation and/or yaw articulation. In embodiments, articulation section 220 is configured to enable both pitch articulation and yaw articulation.

Jaw 284 is pivotably mounted on a distal end portion of support shaft 286 and the drive assembly operably couples clamp trigger 130 of handle assembly 100 with jaw 284 of end effector 280 such that clamp trigger 130 is selectively actuatable to pivot jaw 284 relative to support shaft 286 and blade 282 of end effector 280 from a spaced-apart position to an approximated position for clamping tissue between jaw 284 and blade 282. The drive assembly may include a drive shaft, drive sleeve, drive cables, and/or other suitable components extending through handle assembly 100, elongated shaft 210, articulation section 220, and support shaft 286 to operably couple clamp trigger 130 with jaw 284 and enable pivoting of jaw 284 between the spaced-apart and approximated positions regardless of the articulation of articulation section 220. Jaw 284 includes a more-rigid structural body which is pivotably mounted on a distal end portion of support shaft 286, and a more-compliant jaw liner secured to the more-rigid structural body and positioned to oppose blade 282 to enable clamping of tissue therebetween.

Rotation knob 250 is rotatable in either direction to rotate elongated assembly 200 in either direction relative to handle assembly 100. The articulation assembly may includes gears, pulleys, tension cables, etc. that operably couple articulation knob 260 with the one or more articulation components 222 of articulation section 220 such that rotation of articulation knob 260 manipulates articulating section 220 to thereby articulate end effector 280 relative to elongated shaft 210. Alternatively, articulation knob 260 may be operably coupled to support shaft 286 to induce articulating motion. Additional articulation actuators and/or other suitable articulation actuators are also contemplated.

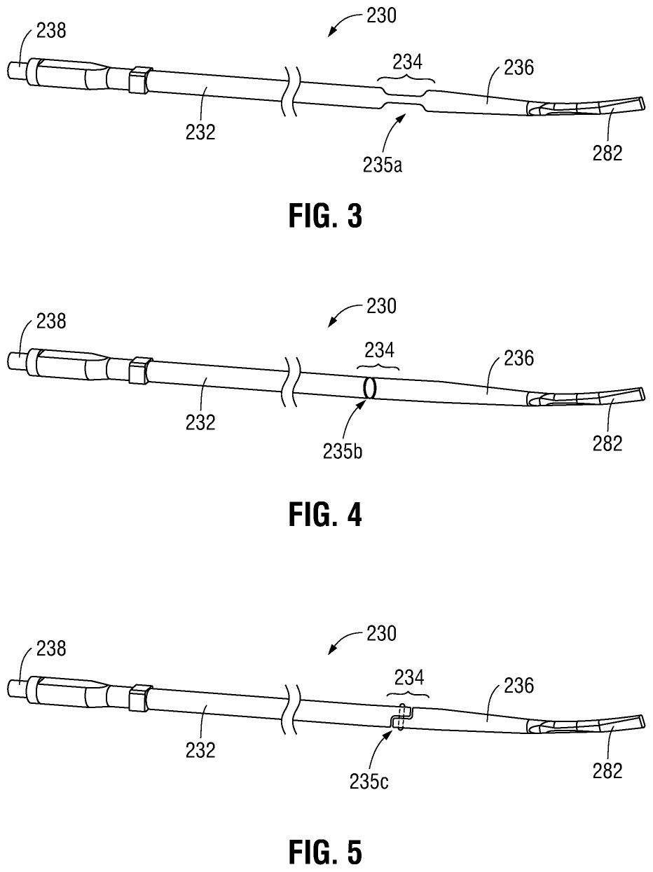

With additional reference to FIG. 3, waveguide 230, as noted above, extends through elongated shaft 210 and articulation section 220. Waveguide 230, more specifically, defines a proximal body portion 232, an articulation portion 234, a distal body portion 236, and blade 282 extending from the distal end of distal body portion 236. Blade 282 serves as the blade of end effector 280. Waveguide 230 extends through elongated shaft 210, articulation section 220, and support shaft 286. Articulation portion 234 of waveguide 230 extends through articulation section 220 of elongated assembly 200 such that, in response to articulation of articulation section 220, articulation portion 234 of waveguide 230 is similarly articulated. Distal body portion 236 extends through support shaft 286 and distally from articulation portion 234. Blade 282 extends distally from distal body portion 236 and support shaft 286 such that blade 282 is positioned to oppose jaw 284 to enable clamping of tissue therebetween. Waveguide 230 may be formed as a single, integral component or may include plural components separately formed and subsequently joined to one another (permanently or releasably) to form waveguide 230.

Blade 282 defines a curved configuration wherein the directions of movement of jaw 284 between the open and clamping positions are perpendicular to the direction of curvature of blade 282. However, it is also contemplated that blade 282 define a straight configuration or that blade 282 curve towards or away from jaw member 284, that is, where the directions of movement of jaw member 284 between the open and clamping positions are coplanar or parallel to the direction of curvature of blade 282.

Waveguide 230 further includes a proximal connector 238 configured for engagement within a corresponding connector of the ultrasonic horn (not shown) of ultrasonic transducer assembly 320 such that ultrasonic vibrations produced by ultrasonic transducer assembly 320 are transmitted along waveguide 230 to blade 282 for treating tissue clamped between blade 282 and jaw 284 or positioned in contact with or close proximity to blade 282.

Referring again to FIGS. 1A and 1B, ultrasonic transducer assembly 320 includes a plurality of piezoelectric elements or other suitable transducer component(s) configured to convert an electrical drive signal into ultrasonic vibration energy for transmission along waveguide 230 to blade 282. Generator assembly 310, powered by battery 400 (or another power source), is configured to generate the electrical drive signal and provide the same to ultrasonic transducer assembly 320. Ultrasonic transducer assembly 320 may be configured to generate any suitable mode of ultrasonic vibration energy for transmission to proximal body portion 232 of waveguide 230, referred to herein as the initial or first mode. More specifically, ultrasonic transducer assembly 320 may be configured to produce a first longitudinal mode of ultrasonic vibration energy, wherein longitudinal ultrasonic vibrations in directions coaxial or parallel with the longitudinal axis of proximal body portion 232 of waveguide 230 are generated and transmitted to proximal body portion 232 of waveguide 230. Alternatively, ultrasonic transducer assembly 320 may be configured to produce a first torsional mode of ultrasonic vibration energy, wherein torsional ultrasonic vibrations rotationally about the longitudinal axis of proximal body portion 232 of waveguide 230 are generated and transmitted to proximal body portion 232 of waveguide 230. Ultrasonic transducer assembly 320 may, as another alternative, be configured to produce a first transverse mode of ultrasonic vibration energy, wherein transverse ultrasonic vibrations in directions perpendicular to the longitudinal axis of proximal body portion 232 of waveguide 230 are generated and transmitted to proximal body portion 232 of waveguide 230. Other suitable first modes of ultrasonic vibration energy, including combination modes of two or more modes, are also contemplated. Further, it is understood that the initial or first mode of ultrasonic vibration energy as utilized herein refers to the primary mode or modes of ultrasonic energy generated at ultrasonic transducer assembly 320 and transmitted to proximal body portion 232 of waveguide 230; it does not include secondary modes generated due to, for example, transducer assembly imperfections, or downstream effects, e.g., as a result of waveguide features, components, material, or forces acting on the waveguide therewith, connections between components, etc. Secondary modes are unintended/unwanted/unavoidable modes or other modes that have vibration amplitudes, in embodiments, that are less than the amplitude of the primary mode or modes and, in other embodiments, that are 15% or less of the amplitude of the primary mode or modes.

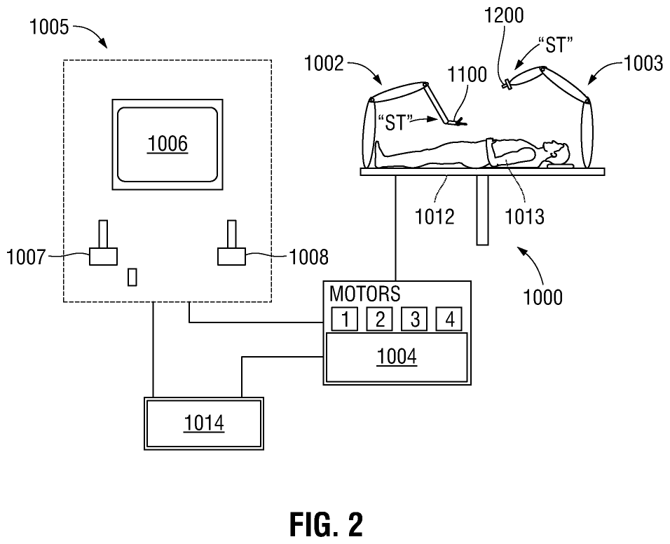

Referring generally to FIG. 2, an embodiment of a robotic surgical system exemplifying the aspects and features of the present disclosure is shown generally identified by reference numeral 1000. For the purposes herein, robotic surgical system 1000 is generally described. Aspects and features of robotic surgical system 1000 not germane to the understanding of the present disclosure are omitted to avoid obscuring the aspects and features of the present disclosure in unnecessary detail.

Robotic surgical system 1000 generally includes a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a person (not shown), for example a surgeon, may be able to telemanipulate robot arms 1002, 1003. Robotic surgical system 1000 may be configured for use on a patient 1013 lying on a patient table 1012 to be treated in a minimally invasive or other suitable manner. Robotic surgical system 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, to which may be attached, for example, a surgical tool “ST” supporting an end effector assembly 1100, 1200. End effector assembly 1100 may be configured as an articulating ultrasonic surgical instrument similarly as detailed above with respect to instrument 10 (FIGS. 1A and 1B) except that robot arm 1002 replaces handle assembly 100 (FIGS. 1A and 1B). End effector 1200 may be any other suitable surgical end effector, e.g., an endoscopic camera, other surgical tool, etc. Robot arms 1002, 1003 may be driven by electric drives, e.g., motors, that are connected to control device 1004. Control device 1004 (e.g., a computer) may be configured to activate the motors, in particular by means of a computer program, in such a way that robot arms 1002, 1003, and, thus, the surgical tools “ST” (including end effectors 1100, 1200) execute a desired movement and/or function according to a corresponding input from manual input devices 1007, 1008, respectively. Control device 1004 may also be configured in such a way that it regulates the movement of robot arms 1002, 1003 and/or of the motors.

Turning to FIGS. 3-5, as noted above, waveguide 230 defines an articulation portion 234 disposed between proximal body portion 232 and distal body portion 236. Articulation portion 234 may include, for example, one or more flexure hinge sections 235a (FIG. 3), a ball-and-socket joint 235b (FIG. 4), one or more pinned joints 235c (FIG. 5), combinations thereof, or other suitable articulation features and/or components that enable articulation of distal body portion 236 and blade 282 of waveguide 230 relative to proximal body portion 232 of waveguide 230.

More specifically, flexure hinge section 235a of waveguide 230 illustrated in FIG. 3 may include at least one section of reduced and/or thinned dimension extending between proximal body portion 232 and distal body portion 236 to enable articulation of waveguide about flexure hinge section 235a in at least one direction. Flexure hinge section 235a, in embodiments where thinned in one dimension (as illustrated), may define a flattened band configuration to enable articulation in directions perpendicular to the large surface area sides of the flattened band. In such embodiments, flexure hinge section 235a may include other portions thinned in other dimensions to enable additional directions of articulation. Alternatively, flexure hinge 235a may define a reduced-diameter cylindrical-shaped configuration to enable articulation in infinite directions, or may define any other suitable configuration. Flexure hinge section 235a need not define reduced dimension portions but may be configured in any other suitable manner to enable flexion thereof to articulate waveguide 230.

Ball-and-socket joint 235b of waveguide 230 illustrated in FIG. 4 may include a ball formed on or engaged with one of proximal body portion 232 or distal body portion 236 and a socket defined within or defined on a component engaged with the other of proximal body portion 232 or distal body portion 236 to enable articulation in any direction via relative rotational sliding movement of the ball within the socket.

Pinned joint 235c of waveguide 230 illustrated in FIG. 5 includes a pivot pin pivotably coupling a distal end portion of proximal body portion 232 and a proximal end portion of distal body portion 236 with one another to enable articulation in directions perpendicular to the longitudinal axis defined through the pivot pin. Additional pinned joints with the same or differently-oriented pivot pins may be provided to enable additional directions of articulation.

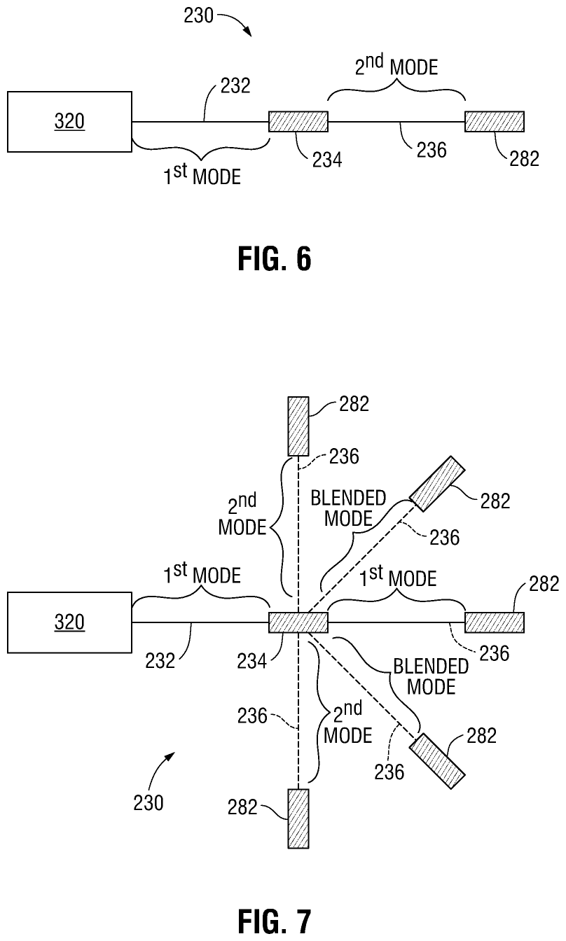

Turning to FIG. 6, in conjunction with FIGS. 1A-5, regardless of the particular articulation portion 234 of waveguide 230 utilized, e.g., flexible waveguide section(s) 235a (FIG. 3), ball-and-socket joint 235b (FIG. 4), pinned joint(s) 235c (FIG. 5), combinations thereof, other suitable articulation portions, etc., articulation portion 234 of waveguide 230 may be configured to convert the ultrasonic vibration energy transmitted therealong from a first mode to a second mode. More specifically, as detailed above, ultrasonic transducer assembly 320 may be configured to produce a first, initial mode of ultrasonic vibration energy that is transmitted from ultrasonic transducer assembly 320 to and along proximal body portion 232 of waveguide 230. The ultrasonic vibration energy is transmitted along proximal body portion 232 of waveguide 230 in the first mode (a primary mode, although secondary modes may also be present, as noted above). Upon reaching articulation portion 234, the configuration of articulation portion 234 serves to convert the ultrasonic vibration energy transmitted therealong from the first mode to a second mode such that the second mode of ultrasonic vibration energy is transmitted along distal body portion 236 to blade 282. In this manner, a first mode of ultrasonic vibration energy is initially generated while a second, different mode of ultrasonic vibration energy is utilized to treat tissue in contact with blade 282.

The first mode, in embodiments, may be a longitudinal mode of ultrasonic vibration energy produced by ultrasonic transducer assembly 320, and the articulation portion 234 may be configured to convert the first, longitudinal mode of ultrasonic vibration energy into a second, torsional and/or transverse mode of ultrasonic vibration energy that is transmitted to blade 282, or vice versa. Any other suitable first and second modes are also contemplated. Further, with respect to combination modes, it is contemplated that mode conversion may include conversion between a combination mode (including plural primary modes) and a single mode (including just one primary mode) and/or change, removal, or addition of one or more modes between a first combination mode and a second combination mode.

Mode conversion may be facilitated, for example, via asymmetric features, force direction-changing features, and/or other suitable features of the articulation portion 234, the distal end portion of proximal body portion 232, and/or the proximal end portion of distal body portion 236. With respect to features incorporated into articulation portion 234, such features may, more specifically, be incorporated into flexible waveguide section(s) 235a (FIG. 3), ball-and-socket joint 235b (FIG. 4), pinned joint(s) 235c (FIG. 5), etc. Mode conversion may additionally or alternatively be facilitated via considering node and antinode locations relative to the articulation portion 234, the distal end portion of proximal body portion 232, and/or the proximal end portion of distal body portion 236.

Turning to FIG. 7, in conjunction with FIGS. 1A-5, regardless of the particular articulation portion 234 of waveguide 230 utilized, e.g., flexible waveguide section(s) 235a (FIG. 3), ball-and-socket joint 235b (FIG. 4), pinned joint(s) 235c (FIG. 5), combinations thereof, other suitable articulation portions, etc., the mode conversion, extent of mode conversion, or lack of mode conversion may vary depending upon on the articulation of articulation portion 234 of waveguide 230. For example, where distal body portion 236 and proximal body portion 232 are aligned with one another, e.g., where articulation portion 234 is un-articulated, the mode of ultrasonic vibration energy transmitted along waveguide 230 may be maintained; that is, the first, initial mode of ultrasonic vibration energy produced by ultrasonic transducer assembly 320 is transmitted along proximal body portion 232, articulation portion 234, and distal body portion 236 to blade 282.

On the other hand, when distal body portion 236 and proximal body portion 232 are disposed at an angle with respect to one another, e.g., where articulation portion 234 is articulated, the mode of ultrasonic vibration energy transmitted along waveguide 230 is at least partially converted from the first mode of ultrasonic vibration energy to a second mode of ultrasonic vibration energy; that is, the first mode of ultrasonic vibration energy produced by ultrasonic transducer assembly 320 is transmitted along proximal body portion 232 to articulation portion 234 where it is at least partially converted into a second mode of ultrasonic vibration energy that is transmitted along distal body portion 236 to blade 282. The amount of conversion may depend upon the articulated position, as detailed below.

For example, where distal body portion 236 and proximal body portion 232 are disposed at an angle of 90 degrees relative to one another, the first mode of ultrasonic vibration energy may be fully converted to the second mode of ultrasonic vibration energy. Where distal body portion 236 and proximal body portion 232 define an angle θ where 0 degrees<θ<90 degrees, the first mode of ultrasonic vibration energy may be partially converted to the second mode of ultrasonic vibration energy such that a blended mode of the first and second modes is transmitted along distal body portion 236 to blade 282. The proportions of the blended mode may depend on the degree of articulation; for example, where θ=45 degrees, the blend may be substantially equal between the first and second modes; where 45 degrees<θ<90 degrees, the blend may include more of the second mode as compared to the first mode, and where 0 degrees<θ<45 degrees, the blend may include more of the first mode as compared to the second mode.

Although specific examples at particular angles are detailed above, the variable mode conversion is not limited thereto; that is, the variable mode conversion need not be linearly proportional to the articulation angle and/or the full first and second modes need not corresponding to angles of 90 degrees and 0 degrees, respectively. Rather, any suitable variable mode conversion may be provided, including configurations where no full (non-blended) mode is provided regardless of articulation angle, and configurations where the articulation angle at which one or both of the full (non-blended) modes occurs is not reachable, e.g., due to constraints on the articulation section 234 of the waveguide 230, the articulation portion 220 of elongated assembly 200, and/or other factors. Variable mode conversion may also include piecewise configuration, e.g., where substantially no blended mode(s) is provided and full conversion between the first and second modes occurs at an articulation angle(s).

In embodiments, the first mode is a longitudinal ultrasonic vibration mode and the second mode is a torsional and/or transverse mode of ultrasonic vibration energy, or vice versa. Other suitable modes including combination modes are also contemplated.

Similarly as detailed above, variable mode conversion may be facilitated, for example, via considering node and antinode locations relative to the articulation portion 234, the distal end portion of proximal body portion 232, and/or the proximal end portion of distal body portion 236, and/or via incorporation of suitable features of articulation portion 234 (e.g., incorporated into flexible waveguide section(s) 235a (FIG. 3), ball-and-socket joint 235b (FIG. 4), pinned joint(s) 235c (FIG. 5), etc.) , the distal end portion of proximal body portion 232, and/or the proximal end portion of distal body portion 236.

It should be understood that various aspects disclosed herein may be combined in different combinations than the combinations specifically presented in the description and accompanying drawings. Further, while several embodiments of the disclosure are presented in the description and accompanying drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An ultrasonic surgical instrument, comprising:
an ultrasonic transducer configured to produce a first mode of ultrasonic vibration energy; and
an ultrasonic waveguide coupled to the ultrasonic transducer and extending therefrom, the ultrasonic waveguide including:
a proximal body portion coupled to and extending distally from the ultrasonic transducer, the proximal body portion configured to receive the first mode of ultrasonic vibration energy from the ultrasonic transducer for transmission therealong;
a distal body portion;
a blade extending distally from the distal body portion; and
an articulation portion interconnecting the proximal and distal body portions, the articulation portion configured to enable articulation of the distal body portion relative to the proximal body portion,
wherein the articulation portion is configured to receive the first mode of ultrasonic vibration energy from the proximal body portion, convert the first mode of ultrasonic vibration energy into a second mode of ultrasonic vibration energy, and transmit the second mode of ultrasonic vibration energy along the distal body portion to the blade.

2. The ultrasonic surgical instrument according to claim 1, further comprising:
a shaft assembly surrounding at least a portion of the ultrasonic waveguide, the shaft assembly including an elongated proximal shaft, an articulating section, and a distal support shaft; and
a jaw pivotably coupled to the distal support shaft,
wherein the proximal body portion of the waveguide extends through the elongated proximal shaft of the shaft assembly, the articulation portion of the waveguide extends through the articulating section of the shaft assembly, the distal body portion of the waveguide extends through the distal support shaft of the shaft assembly, and the blade of the waveguide extends distally from the distal support shaft of the shaft assembly and is positioned to oppose the jaw to enable pivoting of the jaw to clamp tissue between the jaw and the blade.

3. The ultrasonic surgical instrument according to claim 2, wherein the shaft assembly extends distally from a handle assembly.

4. The ultrasonic surgical instrument according to claim 2, wherein the shaft assembly extends distally from a robotic arm.

5. The ultrasonic surgical instrument according to claim 1, wherein the first mode is a longitudinal mode, a torsional mode, a transverse mode, or a combination mode.

6. The ultrasonic surgical instrument according to claim 1, wherein the second mode is a longitudinal mode, a torsional mode, a transverse mode, or a combination mode.

7. The ultrasonic surgical instrument according to claim 1, wherein the articulation portion of the waveguide includes at least one flexure hinge.

8. The ultrasonic surgical instrument according to claim 1, wherein the articulation portion includes a ball-and-socket joint.

9. The ultrasonic surgical instrument according to claim 1, wherein the articulation portion includes at least one pinned joint.

10. An ultrasonic surgical instrument, comprising:
an ultrasonic transducer configured to produce a first mode of ultrasonic vibration energy; and
an ultrasonic waveguide coupled to the ultrasonic transducer and extending therefrom, the ultrasonic waveguide including:
a proximal body portion coupled to and extending distally from the ultrasonic transducer, the proximal body portion configured to receive the first mode of ultrasonic vibration energy from the ultrasonic transducer for transmission therealong;
a distal body portion;
a blade extending distally from the distal body portion; and
an articulation portion interconnecting the proximal and distal body portions, the articulation portion configured to enable articulation of the distal body portion relative to the proximal body portion between an aligned position, wherein the proximal and distal body portions are aligned with one another, and at least one angled position, wherein the proximal and distal body portions are disposed at an angle relative to one another,
wherein, in the aligned position, the articulation portion is configured to receive the first mode of ultrasonic vibration energy from the proximal body portion and transmit the first mode of ultrasonic vibration energy along the articulation portion and the distal body portion to the blade, and
wherein, in the at least one angled position, the articulation portion is configured to receive the first mode of ultrasonic vibration energy from the proximal body portion, at least partially convert the first mode of ultrasonic vibration energy into a different mode of ultrasonic vibration energy, and transmit the different mode of ultrasonic vibration energy along the distal body portion to the blade.

11. The ultrasonic surgical instrument according to claim 10, further comprising:
a shaft assembly surrounding at least a portion of the ultrasonic waveguide, the shaft assembly including an elongated proximal shaft, an articulating section, and a distal support shaft; and
a jaw pivotably coupled to the distal support shaft,
wherein the proximal body portion of the waveguide extends through the elongated proximal shaft of the shaft assembly, the articulation portion of the waveguide extends through the articulating section of the shaft assembly, the distal body portion of the waveguide extends through the distal support shaft of the shaft assembly, and the blade of the waveguide extends distally from the distal support shaft of the shaft assembly and is positioned to oppose the jaw to enable pivoting of the jaw to clamp tissue between the jaw and the blade.

12. The ultrasonic surgical instrument according to claim 11, wherein the shaft assembly extends distally from a handle assembly.

13. The ultrasonic surgical instrument according to claim 11, wherein the shaft assembly extends distally from a robotic arm.

14. The ultrasonic surgical instrument according to claim 10, wherein the first mode is one of: a longitudinal mode, a torsional mode, a transverse mode, or a combination mode, and wherein the different mode is another one of: a longitudinal mode, a torsional mode, a transverse mode, or a combination mode.

15. The ultrasonic surgical instrument according to claim 10, wherein, in at least one of the at least one angled positions, the articulation portion is configured to fully convert the first mode of ultrasonic vibration energy into a second mode of ultrasonic vibration energy as the different mode of ultrasonic vibration energy.

16. The ultrasonic surgical instrument according to claim 10, wherein, in at least one of the at least one angled positions, the articulation portion is configured to partially convert the first mode of ultrasonic vibration energy into a blended mode of ultrasonic vibration energy including both the first mode of ultrasonic vibration energy and a second mode of ultrasonic energy as the different mode of ultrasonic vibration energy.

17. The ultrasonic surgical instrument according to claim 10, wherein:
in a first of the at least one angled positions, the articulation portion is configured to receive the first mode of ultrasonic vibration energy from the proximal body portion, partially convert the first mode of ultrasonic vibration energy into a blended mode of the first mode and a second mode as the different mode of ultrasonic vibration energy, and transmit the different mode of ultrasonic vibration energy along the distal body portion to the blade, and
in a second of the at least one angled positions, the articulation portion is configured to receive the first mode of ultrasonic vibration energy from the proximal body portion, fully convert the first mode of ultrasonic vibration energy into a second mode as the different mode of ultrasonic vibration energy, and transmit the different mode of ultrasonic vibration energy along the distal body portion to the blade.

18. The ultrasonic surgical instrument according to claim 10, wherein an amount of the at least partial conversion depends on the angle in the angled position.

19. The ultrasonic surgical instrument according to claim 10, wherein an amount of the at least partial conversion is proportional to the angle in the angled position.

20. The ultrasonic surgical instrument according to claim 10, wherein the articulation portion of the waveguide includes at least one flexure hinge; a ball-and-socket joint; or at least one pinned joint.
